(19) United States
(12) Patent Application Publication
(10) Pub. No.: US 2017/0360552 A1
(43) Pub. Date: Dec. 21, 2017

(54) Expandable Stent with Constrained End

(71) Applicant: C. R. Bard, Inc., Murray Hill, NJ (US)

(72) Inventor: Mathew Dixon, Mesa, AZ (US)

(73) Assignee: C.R. Bard, Inc., Murray Hill, NJ (US)

(21) Appl. No.: 15/539,567

(22) PCT Filed: Dec. 29, 2015
(86) PCT No.: PCT/US2015/067955

§ 371 Date: Jun. 23, 2017

Related U.S. Application Data
(60) Provisional application No. 62/098,710, filed on Dec. 31, 2014.

(51) Int. Cl. A61F 2/07; A61F 2/95; A61F 2/915

(52) U.S. Cl. CPC A61F 2/07; A61F 2250/0039; A61F 2240/001; A61F 2/915; A61F 2002/072; A61F 2002/9522

(57) ABSTRACT

A stent graft having a tubular stent frame including a plurality of connected struts that form a wall extending along a longitudinal axis from a first end to a second end is described. The stent frame may have a substantially uniform expanded diameter from the first end to the second end, a first expanded polytetrafluoroethylene (ePTFE) covering positioned over an abluminal surface of the tubular stent frame, and a second ePTFE covering positioned over a luminal surface of the tubular stent frame. The second ePTFE covering may be joined to the first ePTFE covering through the tubular stent frame wall at the expanded diameter to form an encapsulated stent. The encapsulated stent may have a reduced diameter section at a first end of the encapsulated stent that is less than the expanded diameter.

### CROSS-REFERENCE TO RELATED APPLICATIONS

This application claims the benefit of U.S. Provisional Application No. 62/098,710, filed Dec. 31, 2014, and titled “Expandable Stent with Constrained End,” which is incorporated herein by reference in its entirety.

### BACKGROUND

Stents are intraluminal prostheses used to maintain, open, or dilate blood vessels. Stent constructions may include lattice type cylindrical frames that define a plurality of openings. Other frameworks for stents include, for example, individual rings linked along the length of the stent by a linking member, a continuous helically wrapped member (that may include one or more linking members), a braid or a mesh formed into a tubular structure, and a series of interconnected struts. Stents may be formed by arranging one or more members in a pattern along a longitudinal axis to define essentially a cylinder and connecting the one or more members or otherwise affixing them in position (e.g., interconnecting with a filament). Stents may also be formed by cutting openings into a tube of material (e.g., shape memory).

Stents may be self-expanding and/or balloon expandable. Self-expanding stents may be delivered to a blood vessel in a collapsed condition and expand in vivo following the removal of a constraining force and/or in the presence of an elevated temperature (due to material properties thereof), whereas balloon expandable stents may be crimped onto a balloon catheter for delivery and require the outwardly directed force of a balloon for expansion. Stents can be made of various metals and polymers and can include a combination of self-expanding and balloon expandable properties.

Synthetic vascular grafts may be used to restore the blood flow in patients suffering from vascular diseases. For example, prosthetic grafts made from expanded polytetrafluoroethylene (ePTFE) may be used to provide favorable patency rates, meaning that the graft maintains an open lumen for the flow of blood therethrough for a beneficial time period. ePTFE includes a microstructure characterized by spaced apart nodes connected by fibrils, the distance between the nodes defined as internodal distance (IND). Grafts may be formed from ePTFE by extruding the ePTFE as a tube or by extruding the ePTFE as a sheet or film that is subsequently fashioned into a tube. Grafts can also be created from fibers woven or knitted into a generally tubular shape.

Stents may be used in combination with vascular grafts or graft material to form stent grafts. Using a biocompatible graft material on a stent can help reduce the inflammatory effect of using a bare metal frame. A bare metal frame may cause inflammation and immune responses that may encourage the re-blocking of a vessel in a condition known as restenosis.

Because stent grafts are often intraluminally deployed in vessels of varying sizes and tortuosity, flexibility can be an important consideration. The flexibility of a stent-graft can be modified in a variety of ways, including by modifying, for example, how the stent is connected to the one or more graft layers, the configuration of the stent and/or graft layer(s), the spacing of the stent struts, rings, or members along the length of the graft(s), etc. U.S. Pat. No. 6,398,803 and U.S. Pat. No. 6,770,087 to Layne et al., which are incorporated by reference in their entirety into this application, describe graft layers with openings to enhance flexibility. Another important consideration in the design of a stent-graft is the ability of the stent to withstand stress and fatigue, caused, for example, by plastic deformations occurring at strut junctions when the stent is subjected to circumferential forces. Stent strength can be enhanced through material choice, stent configuration, arrangement and configuration of graft layers, etc.

When a stent graft is implanted, the stent graft may be expanded to maintain, open, or dilate a blood vessel (e.g., a vein or artery). Implanting a stent graft that has a fully expanded diameter that is larger than the blood vessel diameter is beneficial because a somewhat oversized stent graft is less likely to migrate to an undesired location within the blood vessel after implantation. For example, a stent graft having a 10 mm diameter may be placed in an 8 mm vessel. When implanted, a stent graft with a diameter larger than the diameter of the blood vessel pushes against the blood vessel wall but is also constrained at least somewhat by the blood vessel wall, such that the stent graft does not expand to its full expanded diameter as it would in an unconstrained state. The interaction between the blood vessel wall and the stent graft helps to hold the stent graft in the desired location and prevent unwanted migration in the blood vessel. However, because the stent graft does not expand to its full expanded diameter, the graft material between struts and/or sections of the stent may fold into the interior of the stent graft. When this folding occurs at the ends of the stent-graft (especially at the upstream end), smooth blood flow at the ends of the stent graft is disrupted and turbulence in the blood flow is created at the ends of the stent graft. This disruption/turbulence may lead to and/or facilitate clotting at one or more ends of the stent graft and may ultimately lead to conditions such as graft thrombosis and embolus shedding. Such conditions may lead to critical and even deadly clinical consequences, especially in the brain and heart. The ends of the stent graft tend to be more susceptible to clotting, thrombosis, restenosis, and related issues than the middle of the stent graft. Accordingly, folding of graft material at the ends of the stent graft is much more problematic than folding of graft material in the middle of the stent graft, which is less likely to cause these issues.

In addition to folding at the ends of the stent graft, some reasons the ends of a stent graft may be more likely to suffer from problems related to clotting, thrombosis, and/or restenosis include the abrupt transition from natural blood vessel tissue to the different material of the stent graft and irritation/inflammation caused by interaction of the ends of the stent graft with the blood vessel tissue.

It is believed that reducing and/or eliminating folding at the ends of a stent graft will make a smoother (e.g., less disruptive/turbulent) transition from blood vessel tissue to stent graft material and thereby may reduce incidence of clotting, thrombosis, and/or restenosis associated with the stent graft. It is also believed that reducing the radial force exerted by the stent graft at its ends may reduce the irritation/inflammation of the blood vessel tissue at the ends of the stent graft, which may, in turn, help reduce incidence of clotting, thrombosis, and/or restenosis associated with the stent graft. Reducing the radial force at the ends of the stent graft may also reduce the potential for vessel injury at the ends of the stent graft. Otherwise, in stent grafts of at least some designs, the stent graft ends may damage the vessel wall, such as by causing inflammation and/or tissue perforations in the vessel wall. This damage can lead to serious health complications including infection, hemorrhage, and possible death.

It would be beneficial to have a stent graft that eliminates or reduces folding of the graft material at the ends of the stent graft, reduces potential for vessel injury, and reduces the inflammation/irritation caused by the stent graft, especially at the ends of the stent graft.

### SUMMARY

In one embodiment, a stent graft having a tubular stent frame including a plurality of connected struts that form a wall extending along a longitudinal axis from a first end to a second end is described. The stent frame may have a substantially uniform expanded diameter from the first end to the second end, a first graft covering (e.g., an expanded polytetrafluoroethylene (ePTFE) covering) may be positioned over an abluminal surface of the tubular stent frame. Optionally, a second graft covering (e.g., a second ePTFE covering) may cover a luminal surface of the tubular stent frame. The second graft covering may be joined/bonded/adhered to the first graft covering through interstices or openings in the tubular stent frame wall to form a stent graft with an encapsulated stent. The stent graft may be formed with a reduced diameter section at one or more ends of the encapsulated stent that is less than an expanded diameter of a middle section of the stent graft.

In one embodiment, a method for making a stent-graft includes forming and/or providing an encapsulated stent, including a tubular stent frame covered on an abluminal surface by a first graft covering (e.g., a covering of ePTFE) and on a luminal surface by a second graft covering (e.g., an ePTFE covering), the second graft covering may be bonded/joined/adhered to the first graft covering through interstices or openings in a wall of the tubular stent frame. The stent graft with the encapsulated stent may have an initial diameter. A mandrel may be provided, including a first end having a diameter equal to or less than the initial diameter of the stent graft and a main section having a diameter greater than the initial diameter of the stent graft. The stent graft may be positioned over the mandrel, and the shape of the stent graft may be conformed to the shape of the mandrel.

In one embodiment, a method for making a stent graft includes forming and/or providing an encapsulated stent, including a tubular stent frame covered on an abluminal surface by a first graft covering (e.g., a first ePTFE covering) and on a luminal surface by a second graft covering (e.g., a second ePTFE covering), the second graft covering bonded/joined/adhered to the first graft covering through interstices or openings in a wall of the tubular stent frame. The stent graft may be positioned over an expansion element. The expansion element and stent graft may be placed in a capture tube having a reduced diameter at one or more ends. The expansion element may be expanded to force the encapsulated stent into contact with the capture tube, thereby forming a reduced diameter end on one or more ends of the stent graft.

In one embodiment, a method for making a stent graft includes forming and/or providing an encapsulated stent, including a tubular stent frame covered on an abluminal surface by a first graft material (e.g., a first ePTFE covering) and on a luminal surface by a second graft material (e.g., a second ePTFE covering). The second graft material may be bonded/joined/adhered to the first graft material through interstices or openings in a wall of the tubular stent frame. The stent graft may be positioned over an expansion element at a first diameter. The expansion element may have an expanded second diameter that is greater than the first diameter, and a tapered end having a third diameter greater than the first diameter but less than the second diameter. The expansion element may be expanded to conform the stent graft to the shape of the expansion element.

In one embodiment, a method for treating a patient may comprise obtaining a stent graft, the stent graft including a first end that is constrained to a reduced diameter relative to an expanded diameter of a midsection of the stent graft; inserting the stent graft into a blood vessel; positioning the stent graft in a desired location in the blood vessel; and expanding/deploying the stent graft at the desired location such that the stent graft exerts a radial force outwardly against a wall of the blood vessel, wherein the first end of the stent graft has a new diameter approximately equal to a diameter of the blood vessel at the desired location. Graft material of a graft member/covering of the stent graft may be stretched from the reduced diameter to the larger new diameter during expanding/deploying the stent graft.

These and other embodiments, features and advantages will become more apparent to those skilled in the art when taken with reference to the following more detailed description of the invention in conjunction with the accompanying drawings that are first briefly described.

### BRIEF DESCRIPTION OF THE DRAWINGS

The disclosed devices, components, assemblies, systems and methods can be better understood with reference to the description taken in conjunction with the following drawings, in which like reference numerals identify like elements. The components in the drawings are not necessarily to scale.

FIG. 1 shows a side view of an exemplary stent graft.

FIG. 1A shows a side view representation of a constrained end of an exemplary stent graft.

FIG. 2A shows a side view of an exemplary constrained-end stent graft in a collapsed configuration.

FIG. 2B shows a side view of an exemplary constrained-end stent graft in an expanded configuration.

FIG. 3 shows an exemplary tubular stent frame.

FIG. 4 shows a side view of an exemplary constrained-end stent graft formed by a method of manufacture using a mandrel.

FIG. 5 shows a side view of an exemplary stent graft in an unexpanded first diameter configuration prior to expansion of the midsection of the stent graft and a side view of an exemplary constrained end stent graft having a midsection expanded using an expansion element and ends that remain constrained.

FIG. 6 shows an apparatus including an expansion element and capture tube that may be used in a method of manufacture for a constrained-end stent graft.

FIG. 7A shows an end view of a stent graft without a constrained end implanted in a blood vessel and illustrates in-folding of the graft material.

FIG. 7B shows an end view of an exemplary stent graft having a constrained end implanted in a blood vessel that does not experience in-folding at the constrained end.

FIG. 8 illustrates the reduction of radial force experienced at a constrained end of an exemplary constrained-end stent graft.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DESCRIPTION

The following description should be read with reference to the drawings, in which like elements in different drawings are identically numbered. The drawings, which are not necessarily to scale, depict selected embodiments and are not intended to limit the scope of the invention. The description illustrates by way of example, not by way of limitation, the principles of the invention. Accordingly, the disclosure is not limited to the specific embodiments described. Rather, the inventive principles associated with the embodiments described herein, including with respect to the stent grafts, components, assemblies, systems, methods, etc. described herein, may be applied in a variety of ways, including to other types of devices, components, assemblies, systems, methods, etc. This description will enable one skilled in the art to make and use the invention, and describes several embodiments, adaptations, variations, alternatives and uses of the invention, including what is presently believed to be the best mode of carrying out the invention.

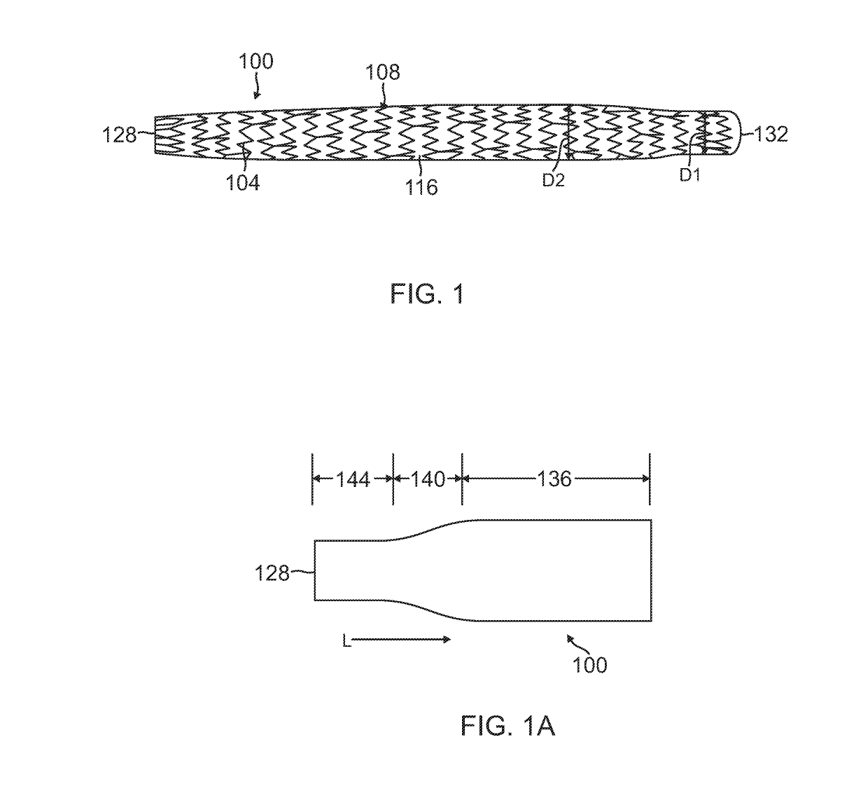

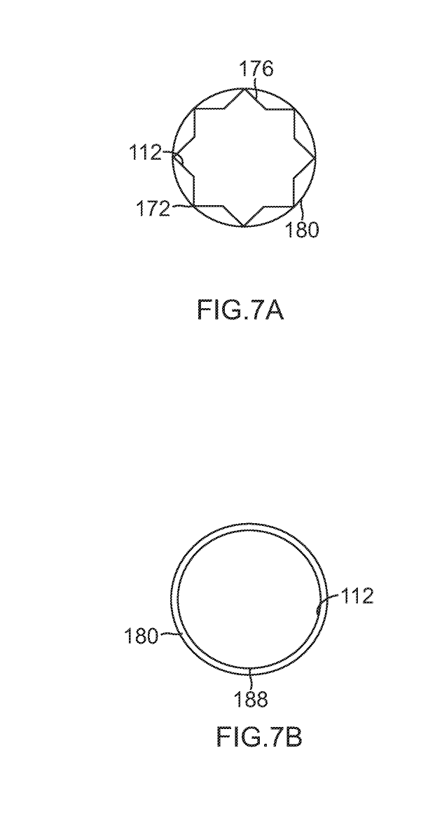

FIG. 1 shows an exemplary embodiment of a stent graft 100. The stent graft 100 may include a stent or stent frame 104, one or more substrates or graft members 116, an outer, abluminal surface 108, an inner, luminal surface 112 (shown in FIGS. 7A and 7B), a first end 128, and a second end 132. As will be discussed in greater detail below, the diameter of the first end 128, second end 132, or both ends of the stent graft may be constrained to have a reduced diameter D1 as compared to a diameter D2 of a middle region of the stent graft. The one or more substrates or graft members 116 may be used to constrain the ends of the stent or stent frame 104 to form the constrained end or ends. Stent graft 100 may be used for insertion entirely or partially into a vasculature of a patient.

In one embodiment, one or both of the first end 128 and the second end 132 of the stent graft 100 may be constrained such that the diameter D1 at the end is less than the diameter D2 at the midsection 136. The first end 128 and/or the second end 132 of the stent graft 100 may be constrained as described in more detail below with respect to methods of manufacture. In one embodiment, both the first end 128 and second end 132 of the encapsulated stent graft may be constrained to the same diameter D1 or may be constrained to different diameters that are each less than D2. In one embodiment, the constrained diameter D1 at one or more ends of the stent graft may have a diameter between approximately 4 mm to 14 mm, 6 mm to 10 mm, or at about 8 mm, and the midsection 136 has a diameter between approximately 6 mm to 18 mm, 8 mm to 14 mm, or at about 10 mm. The midsection 136 may have a uniform diameter or may have a varied diameter across the length of the midsection 136 from the first end 128 to the second end 132. The diameter of the stent graft at the one or more ends of the stent graft may be constrained to a diameter less than any diameter of the midsection 136. The diameter of the first end 128 and/or the second end 132 may be variable and/or constant over different portions thereof. For example, the first end 128 and/or the second end 132 may include a region/section that is constrained into a transition region/section that transitions or tapers from a larger diameter of the midsection 136 to a smaller diameter of the first end 128 or the second end 132. The first end 128 and/or the second end 132 may also include a section/region with an approximately constant diameter. Optionally, the transition region/section may continue to the distal-most tip and/or the proximal-most tip of the first end 128 or the second end 132, such that the diameter of the constrained end or constrained ends varies the entire length of the constrained end or constrained ends, i.e., without any constant diameter region/section.

FIG. 1A shows a side view representation of an exemplary embodiment of a constrained end 128 of an exemplary stent graft 100. The stent graft 100 of FIG. 1A may be the same as or different from the stent graft 100 of FIG. 1. The stent graft may include a larger diameter midsection 136. The constrained end 128 of the stent graft 100 may include a transition section 140, and a reduced/constrained diameter section 144. In one embodiment, the transition section 140 may extend along a longitudinal axis L in a range of approximately 2 mm to 15 mm, 6 mm to 12 mm, or about 10 mm. The transition section may vary in length along a longitudinal axis depending on the use, purpose and design of the stent graft. The reduced/constrained diameter section 144 may have an approximately constant diameter (e.g., varies less than 0.9 mm) or have a diameter that varies somewhat (e.g., varies 1-2 mm). The length of the reduced/constrained diameter section 144 may extend along a longitudinal axis L in a range of approximately 2 mm to 15 mm, 6 mm to 12 mm, or about 10 mm.

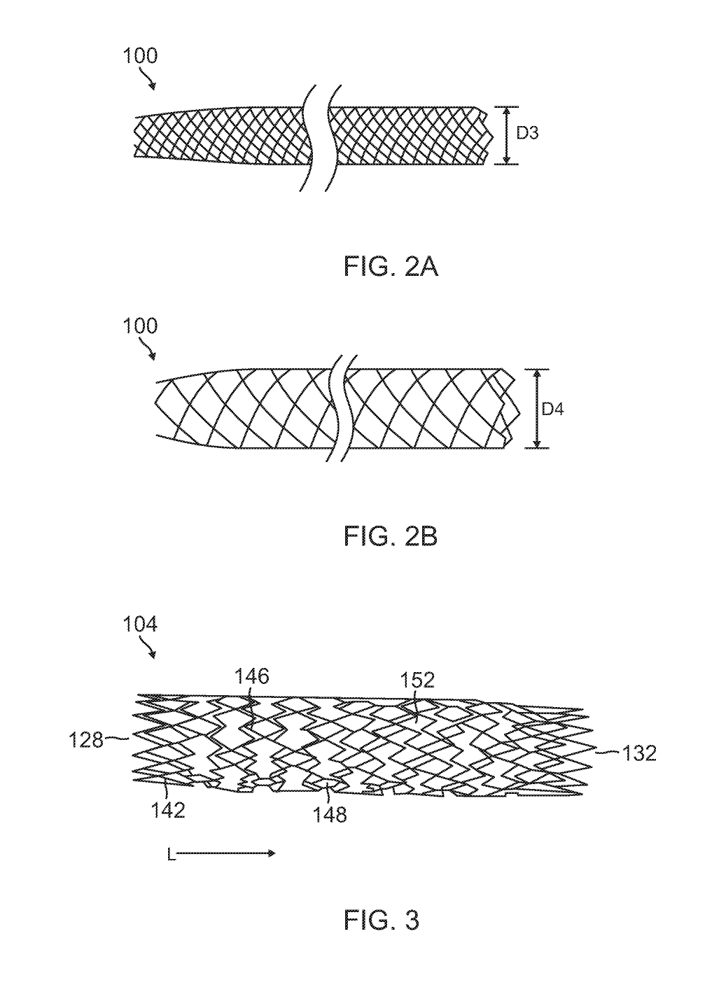

In one embodiment, e.g., as shown in FIGS. 2A, the stent graft 100 may have a first diameter D3 while in a collapsed delivery configuration to assist in the implant delivery procedure, e.g., the diameter D3 is sized to facilitate insertion into vasculature while minimizing trauma to the vasculature and the patient. In one embodiment, the one or more ends may be constrained to a smaller diameter than D3 in the collapsed delivery configuration as shown for example in FIG. 2A. In one embodiment, the one or more constrained ends are at the same or a similar diameter to the D3 diameter of the midsection in the collapsed delivery configuration, e.g., such that in the collapsed delivery configuration, the stent graft appears to have a uniform or generally uniform/constant diameter. In other words, in the collapsed delivery configuration, the midsection of the stent graft may be collapsed to the same or a similar diameter to the one or more constrained ends. Even if the one or more constrained ends appear to have the same or a similar diameter in the collapsed delivery configuration, when in the fully expanded configuration, the one or more constrained ends are constrained to a smaller diameter than the midsection.

As shown in FIG. 2B, the stent graft 100 may have a second diameter D4 in an expanded configuration. The diameter D4 of the expanded configuration being greater that the diameter D3 in the collapsed configuration. The stent graft 100 may be maintained in the collapsed configuration during insertion and positioning of the stent graft in the vasculature. Once the stent graft is properly positioned, the stent graft may be deployed/expanded to an expanded configuration to help open the vasculature or blood vessel and permit blood flow therethrough. In one embodiment, the expanded diameter D4 is about 4 mm to about 18 mm, about 8 mm to about 12 mm, or about 10 mm. The processes/methods described herein may be employed with any stent graft design, including self-expanding stent grafts or balloon expandable stent grafts. The stent graft may be designed to collapse or expand radially in a uniform or non-uniform fashion to assist during delivery.

Stent or stent frame 104 may be of a wide variety of shapes and configurations. In one embodiment, the stent or stent frame 104 is tubular. The stent or stent frame 104 may be cylindrical and have a uniform/constant diameter or a generally uniform/constant diameter (e.g., a diameter that does not vary more than ±1 mm) across the length of the stent or stent frame 104 from end to end or end tip to end tip. FIG. 3 shows an exemplary tubular stent or stent frame 104 embodiment that may include a plurality of circumferential sections 142 forming a wall 148 extending along a longitudinal axis L from a tip of the first end 128 to a tip of the second end 132.

In one embodiment, the stent or stent frame 104 may have a substantially uniform expanded diameter section from the distal-most end (e.g., first end tip) to the proximal-most end (e.g., second end tip). In one embodiment, the stent or stent frame 104 has a non-uniform diameter, e.g., the ends of the stent or stent frame 104 may have a somewhat smaller diameter than the midsection of the stent or stent frame 104. In one embodiment, the stent or stent frame 104 has an open lattice structure, which might comprise for example interstices or openings 152, or bigger slits. In one embodiment, the stent may include a series of struts 146 arranged in various configurations. For example, the stent or stent frame 104 may includes a diamond shape or repeating diamond shape lattice structure. The struts and/or lattice of the stent or stent frame 104 may include geometrically or micro-geometrically deformable shapes, including but not limited to polygons, circles, ovals, triangles, rectangles, squares, and/or the like. The struts may combine end to end to form a repeating zig zag pattern. The struts may form stent rings, e.g., circumferential sections 142 may be stent rings, or may form one or more helical shapes. Circumferential sections 142 formed as stent rings or helical windings may be joined to each other by connectors or bridges.

The structure of the stent or stent frame 104 may permit the stent or stent frame 104 to collapse or expand radially in a uniform or non-uniform fashion. The structure of the stent or stent frame 104 may be formed according to a variety of stent designs, such as, for example, segmented stents, helical stents, solid stents, or combinations thereof. In addition, the stent or stent frame 104 and/or the circumferential sections 142 may be self-expanding or be balloon-expandable, or combinations thereof.

In one embodiment, the stent or stent frame 104 may be wound about an outer surface of a substrate or graft member 116 such that adjacent windings are spaced a distance d from one another. In one embodiment, adjacent stent rings are spaced a distance d apart from each other. In one embodiment, the distance d between adjacent windings or stent rings of the stent or stent frame 104 is approximately equal along the length of the encapsulated stent graft. In one embodiment, the distance d between adjacent windings or stent rings may be varied along the length of the stent graft. For example, beginning at one end of the stent graft, the distance between the first two windings or stent rings, d1, could be less than the distance d2 between subsequent windings or stent rings. The distance between adjacent strut members could then progressively become greater along the length of the stent graft, or could alternate between d1 and d2, etc. In one embodiment, beginning at one end of the stent graft, the distance between the first two windings or stent rings, d1, could be greater than the distance d2 between subsequent windings or stent rings.

The stent or stent frame 104 may include two or more elongate stent members or stent frames combined together. Two or more elongate stent members could be wound about an outer surface of a substrate or graft member 116 in different directions and/or be wound at the same or different angles. In one embodiment, the one or more stent member may be placed under tension as it/they is/are wound about the substrate or graft member 116. If the stent or stent frame 104 is already formed as a tubular or cylindrical stent or stent frame, or a series of connected stent rings, then stent or stent frame 104 may be fitted or slid over the substrate or graft member 116. In one embodiment, no inner substrate or graft member 116 is used, but an outer substrate or graft member 116 is used, e.g., fitted or slid over the stent or stent frame 104.

The stent or stent frame 104 may be formed of a wide variety of materials. For example, the stent or stent frame 104 may be formed of a shape memory material, including, for example, shape memory metals, shape memory alloys, super elastic shape memory metal alloys, linear elastic shape memory alloy, shape memory polymers, and combinations thereof. One preferred shape memory material is Nitinol. The stent or stent frame 104 may also be formed of metals, such as, for example, stainless steel, platinum, and Elgiloy, or certain polymers. The stent or stent frame 104 may also be made of a combination of the materials described herein. In one embodiment, the stent or stent frame 104 may be made of Nitinol. In one embodiment, the stent or stent frame 104 may be cut (e.g., by a laser or other cutter) from a Nitinol tube or sheet. In one embodiment, the stent frame may include a single elongate member or more than one elongate members.

In one embodiment, the luminal surface of the stent or stent frame 104 may be covered by a first substrate or graft member 116 (e.g., a tubular, porous substrate or graft member or an expanded polytetrafluoroethylene (ePTFE) substrate or graft member). The stent or stent frame 104 may be positioned on a radially outward facing surface of the first substrate or graft member 116. The stent graft 100 may also include a second substrate or graft member 116 (e.g., a tubular, porous substrate or graft member or an ePTFE substrate or graft member) extending and covering an abluminal surface of the stent or stent frame 104. The second substrate or graft member 116 may form an outer surface of the stent graft 100. In one embodiment, the stent graft comprises a first substrate or graft member 116 (e.g., a tubular, porous substrate or graft member or an ePTFE substrate or graft member) or first layer thereof, a stent or stent frame 104 positioned on the first substrate or graft member 116, and a second substrate or graft member 116 (e.g., a tubular, porous substrate or graft member or an ePTFE substrate or graft member) or second layer thereof positioned on a radially outward facing surface or abluminal surface of the stent or stent frame 104. The first substrate or graft member 116 and the second substrate or graft member 116 may be made of the same or different materials. In one embodiment, the first substrate or graft member 116 and the second substrate or graft member 116 are both extruded ePTFE tubes. The first substrate or graft member 116 and the second substrate or graft member 116 may be joined, bonded, adhered, or otherwise attached to each other through the interstices or openings 152 of the stent or stent frame 104. This encapsulates the stent or stent frame 104 in between the first substrate or graft member 116 and the second substrate or graft member 116. In one embodiment, the first substrate or graft member 116 and the second substrate or graft member 116 are heated such that they melt slightly together and/or otherwise bond with each other. In one embodiment, a polymeric adhesive, such as polyurethane, may be used to bond the first substrate or graft member 116 to the second substrate or graft member 116. Optionally, the polymeric adhesive can be activated by a solvent, such as tetrahydrofuran (THF). Other modes of attachment (e.g., resin, sutures, heat, pressure, etc.) may also be used to assist in bonding.

The one or more substrates or graft members 116 of the stent-graft described herein may have a thickness in the range between approximately 10 microns and approximately 100 microns, in the range of approximately 20 microns and approximately 60 microns, or in the range of approximately 30 microns and approximately 40 microns. If multiple substrates or graft members 116 are used, the multiple substrates or graft members 116 may be of the same shape, size, and/or thickness or have different shapes, sizes, and/or thicknesses.

Potential materials for the one or more substrates or graft members described herein include, for example, expanded polytetrafluoroethylene (ePTFE), polyester, polyurethane, fluoropolymers, such as perfluoroelastomers and the like, polytetrafluoroethylene, silicones, urethanes, ultra high molecular weight polyethylene, aramid fibers, and combinations thereof. In one embodiment, the substrate or graft member material is ePTFE. In one embodiment, a graft member material may comprise high strength polymer fibers, such as ultra high molecular weight polyethylene fibers (e.g., Spectra®, Dyneema Purity®, etc.) or aramid fibers (e.g., Technora®, etc.). The substrate and/or graft member may include a bioactive agent. In one embodiment, an ePTFE substrate or graft member includes a carbon component along a blood-contacting surface thereof. If multiple substrates or graft members 116 are used, the multiple substrates or graft members 116 may be of the same material or of different materials.

The node-fibril microstructure of one or more ePTFE substrates or graft members used in the stent graft may include various orientations for the fibrils, but in a preferred embodiment, the fibrils are oriented generally parallel to the longitudinal axis of the substrate. The average internodal distance (IND) for one preferred embodiment of a substrate and/or graft described herein is between approximately 6 microns and approximately 80 microns. Also, as described in U.S. Pat. No. 5,790,880 to Banas et al., which is incorporated by reference in its entirety in this application, the substrate and/or graft member may be made of an ePTFE that undergoes nodal elongation during radial expansion.

An ePTFE substrate or graft member may be manufactured in a number of ways, including, for example, extrusion of a tube (seamless), extrusion of a sheet that is subsequently formed into a tube (one or more seams), helical wrapping of ePTFE tape around a mandrel (e.g., multiple seams or preferably a single helical seam), etc. In one embodiment, the method used for forming an ePTFE substrate is by extruding the ePTFE as a seamless tube. It should be appreciated that other forming methods are possible and are within the scope of the invention.

The stent graft described herein may include and/or be utilized with bio-active agents. Bio-active agents can be coated onto a portion or the entirety of the stent and/or graft member for controlled release of the agents once the stent-graft is implanted. The bio-active agents can include, but are not limited to, vasodilator, anti-coagulants, such as, for example, warfarin and heparin. Other bio-active agents can also include, but are not limited to agents such as, for example, anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes—dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

As used herein, the term “bioresorbable” includes a suitable bio-compatible material, mixture of materials or partial components of materials being degraded into other generally non-toxic materials by an agent present in biological tissue (i.e., being bio-degradable via a suitable mechanism, such as, for example, hydrolysis) or being removed by cellular activity (i.e., bioresorption, bioabsorption, or bioresorbable), by bulk or surface degradation (i.e., bioerosion such as, for example, by utilizing a water insoluble polymer that is soluble in water upon contact with biological tissue or fluid), or a combination of one or more of the bio-degradable, bio-erodable, or bio-resorbable material noted above. Potential materials for the stent described herein include, for example, biodegradable polymers such as polylactic acid, i.e., PLA, polyglycolic acid, i.e., PGA, polydioxanone, i.e., PDS, polyhydroxybutyrate, i.e., PHB, polyhydroxyvalerate, i.e., PHV and copolymers or a combination of PHB and PHV (available commercially as Biopol®), polycaprolactone (available as Capronor®), polyanhydrides (aliphatic polyanhydrides in the back bone or side chains or aromatic polyanhydrides with benzene in the side chain), polyorthoesters, polyaminoacids (e.g., poly-L-lysine, polyglutamic acid), pseudo-polyaminoacids (e.g., with back bone of polyaminoacids altered), polycyanocrylates, or polyphosphazenes.

In one embodiment, stent graft 100 may include one or more radiopaque markers for improved visualization of the stent graft in vivo. The radiopaque markers may be arranged along the length of the stent graft and/or at the ends of the stent graft. The stent or stent frame material may itself include a radiopaque material. In one embodiment, the radiopaque material of the markers and/or stent comprises tantalum, gold, platinum, silver, barium sulfate and/or hydroxyapatite, to increase visibility under radio imaging (e.g., x-ray).

Methods of making a stent graft in accordance with the embodiments discussed herein may generally include one or more of the following steps and/or sub-steps (and/or related steps or sub-steps described elsewhere herein):
(1) Forming a stent graft. This may be done in any of the ways discussed herein, e.g., by encapsulating a stent frame in two layers of substrates or graft members. In one embodiment, the stent graft may include a tubular stent frame covered on an abluminal surface by a graft member/covering (e.g., an ePTFE graft member/covering), and on a luminal surface by another graft member/covering (e.g., a different ePTFE graft member/covering). A first graft member/covering may be joined to a second graft member/covering through interstices or openings in a wall of the stent or stent frame, e.g., to encapsulate the stent or stent frame. In one embodiment, the stent graft may be formed with a uniform/constant diameter or generally uniform/constant diameter (e.g., ±1 mm) along its length from end to end or end tip to end tip.
(2) Providing or obtaining a stent graft. In one embodiment, the stent graft may not need to be formed and may only be obtained/provided (such a method does not require the above forming step). The stent graft obtained/provided may include any of the features/characteristics of the stent grafts described herein. The stent graft at this step may have an initial diameter that is smaller relative to its later fully expanded diameter.
(3) Radially expanding a portion of the stent graft to a larger diameter than the initial diameter. This can be done using a number of different methods, including but not limited to expansion via a specially tapered mandrel, expansion using a capture tube and balloon, and expansion using an appropriately sized balloon. The substrate or graft member on the radially expanded portion is stretched such that the stent or stent frame is allowed to expand to either the stent or stent frame's full expanded diameter or a diameter somewhat less than this. This radially expanding step can be done such that one or more ends of the stent graft are not radially expanded and/or are radially expanded to a smaller diameter than the remainder or midsection of the stent graft. Because the one or more ends are not radially expanded, the substrate or graft member material at the one or more ends is not stretched and remains holding the one or more ends constrained to a smaller diameter (e.g., a diameter smaller than the radially expanded/stretched portion).
(4) Radially compressing the stent graft into a radially collapsed delivery configuration with a diameter for insertion into a vasculature. The stent graft may be radially compressed onto a balloon of a balloon catheter, such that the balloon may be expanded to expand and implant the stent graft. The stent graft may be radially compressed and loaded into a delivery sheath of a delivery catheter that may be retracted to deliver the stent graft.

In one embodiment, two substrates or graft members/coverings may be joined/bonded/adhered to one another while one of an abluminal graft member/covering and a luminal graft member/covering is compressed and the other is placed under tension. For example, a luminal graft member/covering 116 may be axially/longitudinally compressed in a range of approximately 50% to approximately 97% of its original, uncompressed length. While the luminal surface covering 116 is held in an axially/longitudinally compressed state, the stent or stent frame 104 may be positioned over an outer surface of the luminal graft member/covering 116. The stent or stent frame 104 may optionally include a coating of polycarbonate urethane. Once the stent or stent frame 104 is in the predetermined position over the luminal graft member/covering 116, the abluminal graft member/covering 116 may be positioned over the stent or stent frame 104 and longitudinally compressed luminal graft member/covering 116. The abluminal graft member/covering 116 may then be placed under tension (e.g., proximal and distal ends of the luminal surface covering may be pulled in opposite directions) and clamped or otherwise fixed in place over the stent or stent frame 104 and compressed luminal graft member/covering 116. In this tensioned state, the material of the luminal surface covering 118 may cover substantially all of, or only a portion of, an outer surface of the stent or stent frame. The stent graft 100 in its assembled form may then be contacted with a polymeric adhesive, such as polyurethane, to bond the luminal graft member/covering to the stent frame and/or the abluminal graft member/covering. Optionally, the polymeric adhesive can be activated by a solvent, such as tetrahydrofuran (THF). Other modes of attachment (e.g., resin, sutures, heat, pressure, etc.) may also be used in conjunction with the solvent to assist in bonding.

In one embodiment, luminal graft member/covering may be positioned to cover the luminal surface of the stent or stent frame 104. An abluminal graft member/covering may be positioned to cover the abluminal surface of the stent or stent frame 104. The luminal graft member/covering and the abluminal graft member/covering may be joined/bonded/attached using the application of heat and/or pressure, and/or other methods. Adhesives and/or solvents may also be used instead of, or in conjunction with, the aforementioned attachment methods.

For example, a coating, such as urethane resin, could be disposed on the abluminal and/or luminal surfaces of the tubular stent frame 104 to contact the substrates or graft members 116 disposed thereon when assembled together. Thereafter, the assembly may be soaked in a solvent for bonding. In one embodiment, the stent or stent frame 104 may be sutured to the substrate at various locations along the length thereof. In one embodiment, the substrate is initially unsintered ePTFE and is located over a mandrel for positioning of the tubular stent frame, which may be sintered or partially sintered. In one embodiment, the assembly is then heated to sinter the first substrate or graft member 116 to the second substrate or graft member 116 (e.g., 360 degrees C. for 10 minutes). Prior to heating, the assembly may be subject to pressures to force the separate layers together (e.g., by wrapping with a tape). In one embodiment, the luminal graft member/covering is joined to the abluminal graft member/covering through openings or interstices in the stent or stent frame wall 148 at the expanded diameter D2 to encapsulate the stent or stent frame 104.

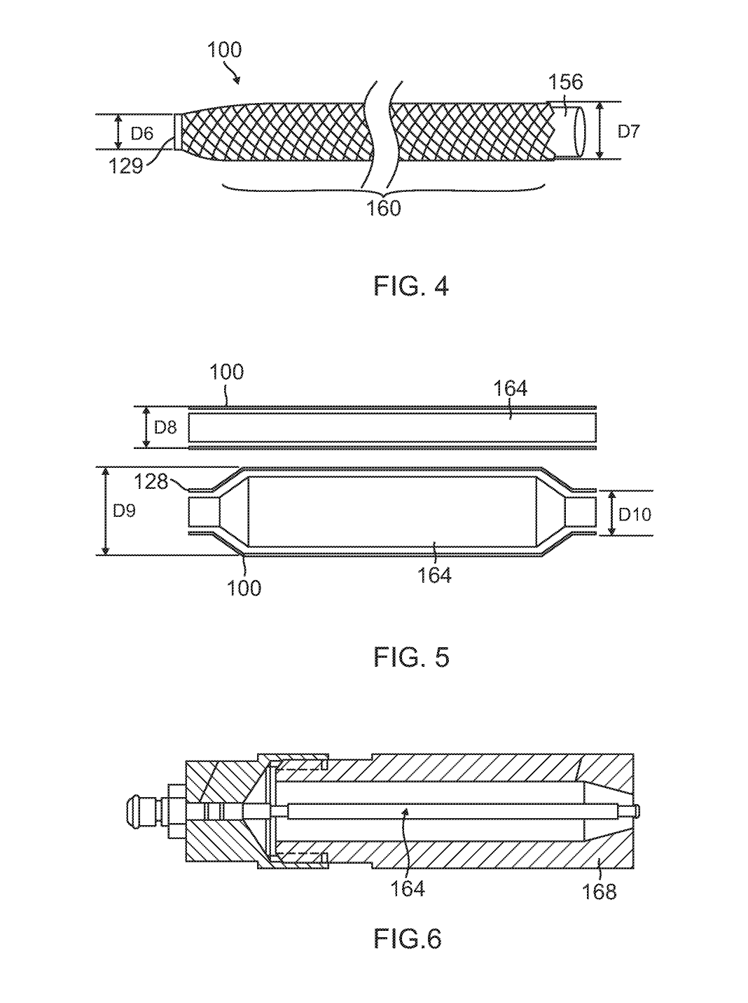

As shown in FIG. 4, a stent graft 100 having an unexpanded first diameter may be positioned over a mandrel 156. The mandrel 156 may include a first end 129 having a diameter D6 (which may be the same as, slightly less than, or somewhat larger than the first diameter), and a main section 160 having a second diameter D7 greater than the first diameter and greater than the diameter D6 of the first end. The second diameter D7 can be as large as or slightly less than the fully expanded diameter of the stent or stent frame 104. A portion of the stent graft may be stretched from the unexpanded first diameter to the second diameter D7 as the stent graft is loaded onto the mandrel 156. The stent graft may conform to the shape of the mandrel. An end (e.g., end 128 or end 132) of the stent graft may be positioned over the first end 129 of the mandrel without passing over the larger diameter main section of the mandrel 156, such that the end of the stent graft is not stretched to the second diameter D7. The end of the stent graft may be stretched to the diameter D6 (or not stretched if D6 is the same size or smaller than the unexpanded first diameter of the stent graft). A transition section/region may be formed that transitions the diameter of the stent graft from a smaller constrained diameter up to the diameter D7. The graft material in the region of the stent graft 100 placed over the main section of the mandrel 156 is stretched to approximately the diameter D7, while the graft material of the end of the stent graft that is not passed over the main section of the mandrel 156 is not stretched to D7, and the graft material at the end of the stent graft thereby constrains the end of stent or stent frame 104 and the end of the stent graft 100 to a diameter smaller than D7 (e.g., to a diameter D6). The mandrel 156 may be implemented in varying designs to achieve different sizes and/or shapes as desired. In one embodiment, a collapsible mandrel may be used such that one or both ends of the stent graft 100 may have a reduced diameter as compared to the radially expanded diameter section.

FIG. 5 shows a side view of an exemplary stent graft in an unexpanded first diameter configuration prior to expansion of the midsection of the stent graft and a side view of an exemplary constrained end stent graft having a midsection expanded using an expansion element and ends that remain constrained. In one embodiment, the stent graft 100 may be positioned over an expansion element 164 while having an unexpanded first diameter D8. Various types of expansion elements, including angioplasty balloons or other balloons, may be used. The expansion element 164 may be radially expanded such that at least a portion of the stent graft (e.g., a midsection) is expended to an expanded second diameter D9 that is greater than the first diameter. One or more ends of the stent graft may not be expanded or may be only partially expanded. For example, a tapered end 128 may have a third diameter D10 greater than the unexpanded first diameter, but less than the expanded second diameter. Optionally, the diameter D10 may be the same as or similar to the diameter D8. The stent graft may conform or partially conform to a shape of or a shape of a portion of the expansion element 164 as it is radially expanded. Configurations of a stent graft with one or more constrained ends similar to those described elsewhere herein may be formed using an expansion element as described herein. The graft material in the midsection of the stent may be stretched to an expanded diameter that is the same, similar, or somewhat less than the full expanded diameter of the stent or stent frame 104, while the graft material at one or more ends of the stent graft is not stretched or is stretched to a lesser extent, such that the graft material continues to constrain the one or more ends of the stent or stent frame 104 to a reduced diameter.

FIG. 6 shows an apparatus including an expansion element and capture tube that may be used in a method of manufacture for a constrained-end stent graft. A method of manufacturing a stent graft using the apparatus in FIG. 6 may be similar to the method discussed above with respect to FIG. 5, but may also use a capture tube 168 to help shape the stent graft as desired. The stent graft 100 having an unexpanded first diameter (e.g., the same as or similar to D8 shown in FIG. 5) may be positioned over an expansion element 164, such as a balloon. Various types of expansion elements, including angioplasty balloons or other balloons or expansion elements described herein, may be used. The stent graft and expansion element 164 may be enclosed in a capture tube 168. The capture tube 168 may have a variety of shapes, and may include a reduced diameter at one or more ends of the capture tube 168. The expansion element 164 may be expanded inside the capture tube 168, and thereby radially expand and force the stent graft 100 into contact with one or more inner surfaces of the capture tube. In this way, the graft material of the stent graft may be stretched such that the stent graft has a configuration the same as or similar to the interior of the capture tube 168. Configurations of a stent graft with one or more constrained ends similar to those described elsewhere herein may be formed in this manner. The graft material in the midsection of the stent may be stretched to an expanded diameter (e.g., the same as or similar to D9 shown in FIG. 5) that is the same, similar, or somewhat less than the full expanded diameter of the stent or stent frame 104, while the graft material at one or more ends of the stent graft is not stretched or is stretched to a lesser extent, such that the graft material continues to constrain the one or more ends of the stent or stent frame 104 to a reduced diameter (e.g., to a diameter the same as or similar to D10 in FIG. 5).

In embodiments having one or more constrained ends, less substrate and/or graft material 116 may be required at the constrained first end and/or constrained second end of the stent graft (e.g., because the graft material is not stretched or expanded to the same extent as the graft material at the midsection of the stent graft), at least as compared to an expanded stent graft having a substantially uniform diameter. In one embodiment, less ePTFE material is required at a constrained end of a stent graft. In one embodiment, less ePTFE material is required at both a constrained first end and a constrained second end of a stent graft. Although, the use of less material is not required, i.e., the amount of graft material may be uniform from end to end of the stent graft, but the graft material at the ends may not be stretched as much as graft material at the midsection.

As discussed above, a stent graft having a fully expanded diameter that is larger than the blood vessel diameter may be implanted in the blood vessel. For example, a stent graft having a 10 mm diameter may be placed in an 8 mm vessel. When implanted, a stent graft with a diameter larger than the diameter of the blood vessel pushes against the blood vessel wall but is also constrained at least somewhat by the blood vessel wall, such that the stent graft does not expand to its full expanded diameter as it would in an unconstrained state. Because the stent graft does not expand to its full expanded diameter, the graft material between struts and/or sections of the stent may fold into the interior of the stent graft.

FIG. 7A shows an end view of a stent graft without a constrained end implanted in a blood vessel and illustrates in-folding of the graft material at the unconstrained end. A stent or stent frame 172 (e.g., which may be the same as or similar to the stent or stent frame 104 discussed herein) with a substantially uniform shape having no constrained ends tends to experience in-folding of graft material 176 (e.g., may be the same as or similar to material of the one or more substrates or graft members 116 discussed herein) after being deployed in a vessel 180 of a patient's body. The outwardly facing points of FIG. 7A represent regions of the stent graft including the stent or stent frame 172, whereas the inwardly pointing portions of FIG. 7A represent graft material that has folded inwardly, e.g., in the interstices or open regions between portions or struts of the stent or stent frame 172. When in-folding occurs at the ends of the stent-graft (especially at the upstream end), smooth blood flow at the ends of the stent graft is disrupted and turbulence in the blood flow is created at the ends of the stent graft. This disruption/turbulence may lead to and/or facilitate clotting or emboli formation at one or more ends of the stent graft and may ultimately lead to conditions such as graft thrombosis and embolus shedding. The ends of the stent graft tend to be more susceptible to clotting, thrombosis, restenosis, and related issues than the middle of the stent graft. Accordingly, folding of graft material at the ends of the stent graft is much more problematic than folding of graft material in the middle of the stent graft, which is less likely to cause these issues.

It is believed that reducing and/or eliminating folding at the ends of a stent graft will make a smoother (e.g., less disruptive/turbulent) transition from blood vessel tissue to stent graft material and thereby may reduce incidence of clotting, emboli formation, thrombosis, and/or restenosis associated with the stent graft. It is also believed that reducing the radial force exerted by the stent graft at its ends (e.g., by constraining the ends with the graft material to a constrained diameter) may reduce the irritation/inflammation of the blood vessel tissue at the ends of the stent graft, which may, in turn, help reduce incidence of clotting, emboli formation, thrombosis, and/or restenosis associated with the stent graft.

FIG. 7B shows an end view of an exemplary stent graft 188 (e.g., the same as or similar to the stent grafts 100 described herein) having at least one constrained end implanted in a blood vessel 180 that does not experience in-folding at the constrained end. FIG. 7B shows that the stent graft with constrained end does not experience the same in-folding as the stent graft of FIG. 7A. When the stent graft is deployed/implanted in the blood vessel 180, a balloon (e.g., an angioplasty balloon) may be used to expand the stent graft to its implanted configuration. When the delivery balloon is expanded, the stent graft is expanded and the graft material at the constrained ends may be stretched to approximately the diameter of the blood vessel, such that the graft material helps hold the stent graft end at the diameter of the blood vessel. As the one or more ends of the stent graft are stretched to the diameter of the blood vessel, there is no or very little excess graft material that can fold in. Further, the graft material continues to constrain the one or more ends of the stent graft, such that the one or more constrained ends contact the blood vessel walls with a smaller or reduced radial force as compared to an unconstrained end or the midsection of the stent graft. This reduced radial outward force at the ends helps limit the body's reaction or response to the stent graft at the one or more constrained ends, e.g., reducing irritation and inflammation.

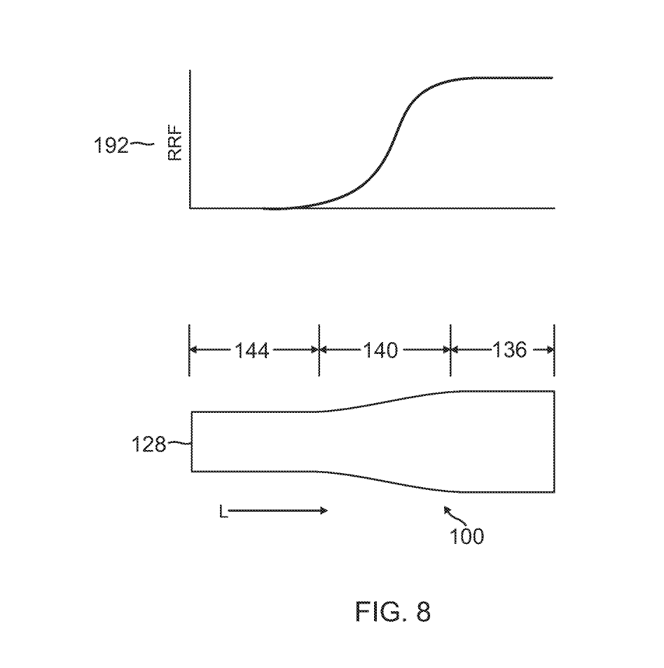

FIG. 8 illustrates the reduction of radial force experienced at a constrained end of an exemplary constrained-end stent graft. The analytical model/graph 192 shown in FIG. 8 corresponds to a constrained-end stent graft 100. As shown in FIG. 8, a stent graft 100 having a constrained end exerts only limited radial force on the blood vessel at and near the constrained end of the stent graft. This radial force is significantly reduced relative to stents and stent grafts with unconstrained ends. As shown in the analytical model/graph 192, the theoretical radial force experienced in a vessel is low at the reduced or constrained diameter section 144. At the transition section 140, the theoretical radial force increases smoothly, rather than abruptly. The radial force is greater at the expanded diameter section 136 than at transition section 140 or constrained diameter section 144. The reduced radial force outward on the vessel at the ends helps reduce the risk of injury to the vessel and reduce irritation of the tissue. This helps protect the vessel and helps moderate or reduce the body's reaction to the implanted stent graft.

Methods of using the stent grafts described herein (e.g., to treat a patient, to treat a stenosis, to open or expand a portion of a blood vessel) may include one or more of the following steps and/or sub-steps (and/or related steps or sub-steps described elsewhere herein):
(1) Obtaining a stent graft. The stent graft may include any of the properties/characteristics of the stent grafts described herein. A first end of the stent graft may be constrained to a first reduced diameter relative to an expanded diameter of a midsection of the stent graft. A second end of the stent graft may be constrained to a second reduced diameter relative to the expanded diameter of a midsection of the stent graft. The first and/or second ends may have a smaller diameter than any portion of the midsection that extends from the first end to the second end. The first end and/or the second end may be constrained by graft material of a graft member/covering positioned along a portion or all of a stent or stent frame.
(2) Inserting the stent graft into a blood vessel. This can be done using a delivery catheter designed for delivery of stents and/or stent grafts. The delivery catheter may include an outer sheath that is retracted to allow the stent graft to expand. The delivery catheter may include an expandable element, e.g., a balloon that may be inflated/expanded to expand the stent graft to a deployed/expanded/implanted configuration.
(3) Positioning the stent graft in a desired location in the blood vessel. For example, positioning the stent graft across a stenosis or otherwise narrowed region of a vessel of a body of a patient.
(4) Expanding/deploying the stent graft at the desired location. For example, expanding/deploying the stent graft such that the stent graft exerts a radial force outwardly against a wall of the blood vessel. The stent graft may be expanded/deployed such that the constrained first end of the stent graft has a third diameter approximately equal (e.g., ±1 mm or equal to 1 mm larger) to a diameter of the blood vessel at the desired location (e.g., a natural vessel diameter just prior to or after the narrowed region or stenosis). If the stent graft includes a constrained second end, the stent graft may be expanded/deployed such that the constrained second end of the stent graft has a fourth diameter approximately equal (e.g., ±1 mm or equal to 1 mm larger) to a diameter of the blood vessel at the desired location (e.g., a natural vessel diameter just prior to or after the narrowed region or stenosis). The first reduced diameter and the third diameter may be equal or the third diameter may be larger than the first reduced diameter. The second reduced diameter and the fourth diameter may be equal or the fourth diameter may be larger than the second reduced diameter. Where the graft material of the graft member/covering constrains the first end, the graft material (e.g., ePTFE) may be stretched during expanding/deploying the stent graft from the first reduced diameter to the third diameter. Similarly, where the graft material of the graft member/covering constrains the second end, the graft material (e.g., ePTFE) may be stretched during expanding/deploying the stent graft from the second reduced diameter to the fourth diameter. Even in an expanded/deployed/implanted configuration, the first end and/or the second end may be constrained (e.g., the graft material may exert a constraining force holding the stent or stent frame and stent graft to a constrained state, e.g., with a diameter at the third diameter and/or fourth diameter).

For purposes of this disclosure, “permanently joined” means that two or more objects are connected such that the process of disconnecting the objects would damage at least one of the objects.

For purposes of this disclosure, “securely joined” means that two or more objects are connected such that the objects cannot move with respect to one another without the objects disconnecting.

For purposes of this disclosure, “adhered” means that two or more objects are connected such that one or more of the objects can slide along the surface of another object without the sliding surfaces disconnecting.

Two values are substantially equal when those of ordinary skill in the art would not consider swapping the values likely to meaningfully change the inventions operation.

The above devices, components, systems, assemblies, methods, etc. have generally been described as being applied to a stent graft for insertion into vasculature or a blood vessel; however, the principles described may be applied to other types of devices, components, systems, assemblies, methods, etc. For example, the stent grafts described herein may be used in bodily vessels other than blood vessels. Further, the features described in one embodiment herein may generally be combined with features described in other embodiments herein. All of the devices, components, systems, assemblies, methods, etc. disclosed and claimed herein may be made and executed without undue experimentation in light of the present disclosure.

While the devices, components, systems, assemblies, methods, etc. of this invention may have been described in terms of particular variations and illustrative figures, it will be apparent to those skilled in the art that the invention is not so limited and that variations may be applied to the devices, components, systems, assemblies, methods, etc. For example, with respect to the methods, uses, and/or steps described herein variations may occur in the steps, uses, the sequence/order of steps, etc. described herein without departing from the concept, spirit, and scope of the invention, as defined by the claims. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A stent graft comprising:
a cylindrical wall;
a first tube disposed coaxially outside of the wall; and
a second tube disposed coaxially inside of the wall connected to the first tube through or around the wall at one or more locations,
wherein at least one end region of the stent graft exhibits a diameter smaller than a diameter of a middle region of the stent graft.

2. The stent graft according to claim 1, wherein the locations are permanently joined, securely joined, or adhered.

3. The stent graft according to claim 2, wherein the end diameters of the stent graft are or are not substantially equal.

4. The stent graft according to claim 3, wherein the smaller diameter end region is about 8 mm and the middle region diameter is about 10 mm.

5. The stent graft according to claim 4, wherein the smaller diameter end region is about 10 mm long.

6. The stent graft according to claim 5, further comprising a 4-10 mm long section transitioning the end region to the middle region.

7. The stent graft according to claim 6, wherein none of the tube ends exhibit in-folding.

8. The stent graft according to claim 7, wherein at least one of the first tube ends or the second tube ends exhibits a diameter smaller than either a first tube middle region diameter or a second tube middle region diameter.

9. The stent graft according to claim 2, wherein the tube end exhibiting a smaller diameter constrains a corresponding end of the wall to a diameter smaller than a diameter of the middle region of the wall.

10. The stent graft according to claim 9, wherein one first tube end and a corresponding second tube end exhibit a diameter small than either a first tube middle region diameter or a second tube middle region diameter.

11. The stent graft according to claim 10, wherein both first tube ends and both second tube ends exhibit a diameter small than either a first tube middle region diameter or a second tube middle region diameter.

12. The stent graft according to claim 2, wherein at least one wall end constrains a corresponding tube end to a diameter smaller that a diameter of the middle region of the wall.

13. The stent graft according to claim 12, wherein one first tube end and a corresponding second tube end exhibit a diameter small than either a first tube middle region diameter or a second tube middle region diameter.

14. The stent graft according to claim 13, wherein both first tube ends and both second tube ends exhibit a diameter small than either a first tube middle region diameter or a second tube middle region diameter.

15. A method for making a stent graft comprising:
making an assembly by
providing a stent having a first diameter;
placing a first tube coaxially outside of the stent;
placing a second tube coaxially inside of the stent; and
connecting the first tube to the second tube through or around the stent at one or more locations;
placing the assembly on a mandrel having a first end diameter smaller than and a middle region diameter larger than the first diameter; and
conforming the assembly shape to the mandrel shape

16. The method according to claim 15, wherein the conforming step comprises:
placing the assembly under tension;
followed by
wrapping ePTFE tape over the assembly.

17. The method according to claim 16, wherein the mandrel has a taper region between the first end and the middle region and wherein the taper diameter is between the diameter of the first end and of the diameter of the middle region.

18. A method for making a stent graft comprising:
making an assembly by
providing a stent having a first diameter;
placing a first tube coaxially outside of the stent;
placing a second tube coaxially inside of the stent; and
connecting the first tube to the second tube through or around the stent at one or more locations;
positioning the assembly over an expansion element having a first end diameter smaller than and a middle region diameter larger than the first diameter;
placing the expansion element and the assembly in a capture tube having a reduced diameter at one end; and
conforming the assembly to the expanded expansion element.

19. The method according to claim 18, wherein the conforming step comprises expanding the expansion element to push the assembly against the capture tube.

20. The method according to claim 19, wherein the expansion element has a taper region between the first end and the middle region and wherein the taper diameter is between the diameter of the first end and of the diameter of the middle region.
